(19) United States
(12) Patent Application Publication
(10) Pub. No.: US 2023/0050381 A1
(43) Pub. Date: Feb. 16, 2023

(54) METHOD FOR DETECTING THERMAL ANOMALY IN COMPOSITE STRUCTURE

(71) Applicants: Agency for Science, Technology and Research, Singapore (SG); ST Engineering Aerospace Ltd, Singapore (SG)

(72) Inventors: Chun Yong, Andrew NGO, Singapore (SG); Vinod Kumar S/O SIVARAJA, Singapore (SG); Jun Yuan James TAN, Singapore (SG); Joshua VANU, Singapore (SG)

(21) Appl. No.: 17/797,981

(22) PCT Filed: Nov. 6, 2020
(86) PCT No.: PCT/SG2020/050639

§ 371 Date: Aug. 5, 2022

(30) Foreign Application Priority Data
Feb. 7, 2020 (SG) 10202001126S

(51) Int. Cl. G01N 25/72

(52) U.S. Cl. CPC G01N 25/72; G01N 2033/0003

(57) ABSTRACT

A method (10) for detecting a thermal anomaly in a composite structure (12) is provided. The method (10) includes radiatively heating (14) the composite structure (12) for a period of between about 15 seconds (s) and about 25 s, cooling (24) the heated composite structure (12), monitoring (26) temperature changes of the composite structure (12) as the composite structure (12) cools, and generating (30) a thermal image of the composite structure (12) based on the temperature changes.

### FIELD OF THE INVENTION

The present invention relates, in general, to active infrared thermography (IRT) and, more particularly, to a method for detecting a thermal anomaly in a composite structure.

### BACKGROUND OF THE INVENTION

Active infrared thermography (IRT) methods are currently used to detect thermal anomalies in honeycomb composite structures during aircraft maintenance.

Drawbacks of current active IRT methods for thermal anomaly detection include a requirement for close heating of a composite surface and detection difficulties when a thermal anomaly is located under thick paint and/or a decal.

It would therefore be desirable to provide a thermal anomaly detection method that allows greater flexibility in positioning of components and that can overcome composite surface restrictions.

### SUMMARY OF THE INVENTION

Accordingly, in a first aspect, the present invention provides a method for detecting a thermal anomaly in a composite structure. The method includes radiatively heating the composite structure for a period of between about 15 seconds (s) and about 25 s, cooling the heated composite structure, monitoring temperature changes of the composite structure as the composite structure cools, and generating a thermal image of the composite structure based on the temperature changes.

Other aspects and advantages of the invention will become apparent from the following detailed description, taken in conjunction with the accompanying drawings.

### BRIEF DESCRIPTION OF THE DRAWINGS

Embodiments of the invention will now be described, by way of example only, with reference to the accompanying drawings, in which:

FIG. 1 is a schematic flow diagram illustrating a method for detecting a thermal anomaly in a composite structure in accordance with an embodiment of the present invention;

FIG. 2 is a schematic diagram illustrating a set-up for implementing the method of FIG. 1;

FIGS. 3A and 3B are schematic diagrams illustrating set-ups for implementing the method of FIG. 1;

FIG. 4 illustrates a comparison between thermal images of various surfaces obtained using a conventional active infrared thermography (IRT) method and those obtained using the method of FIG. 1;

FIG. 5 illustrates a comparison between thermal images of a sample surface obtained using the method of FIG. 1 with different heating durations and a visual image of the sample surface;

FIG. 6 contains thermal images of another sample surface obtained using the method of FIG. 1 under different conditions and settings; and

FIG. 7 is a plot of a maximum rise in temperature as a function of heating duration and distance.

### DETAILED DESCRIPTION

The detailed description set forth below in connection with the appended drawings is intended as a description of presently preferred embodiments of the invention, and is not intended to represent the only forms in which the present invention may be practiced. It is to be understood that the same or equivalent functions may be accomplished by different embodiments that are intended to be encompassed within the scope of the invention.

The term “about” as used herein refers to both numbers in a range of numerals and is also used to indicate that a value includes the standard deviation of error for the device or method being employed to determine the value. The term “about” as used herein can allow for a degree of variability in a value or range, for example, within 10%, within 5%, or within 1% of a stated value or of a stated limit of a range.

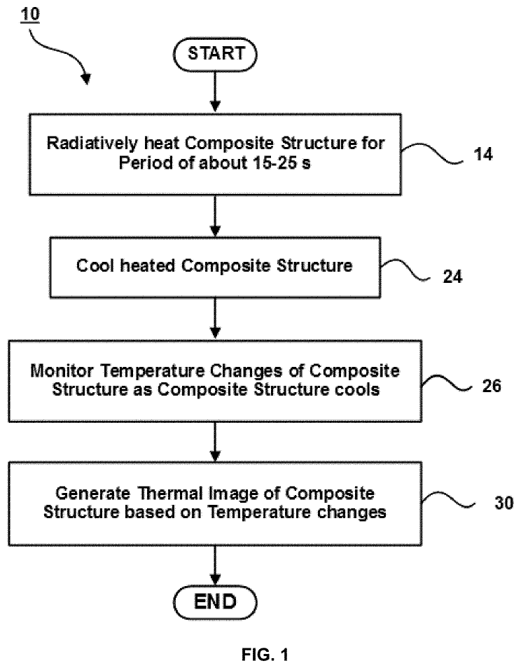

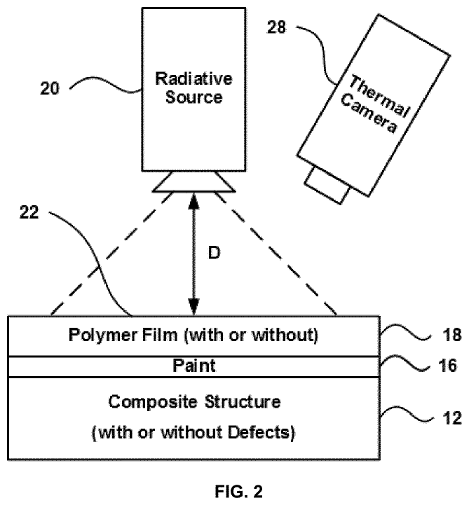

With reference to FIGS. 1 and 2, a method 10 for detecting a thermal anomaly in a composite structure 12 will now be described. The method 10 begins at step 14 by radiatively heating the composite structure 12 for a period of between about 15 seconds (s) and about 25 s.

The composite structure 12 may have a honeycomb structure and may include a layer of paint 16 and, in some instances, a polymer film 18 such as, for example, a decal on top of the layer of paint 16. The layer of paint 16 may have a thickness of between about 250 microns (μm) and about 750 μm.

Advantageously, radiative heating of the composite structure 12 is faster and less susceptible to environmental factors than convective heat transfer. Further advantageously, use of the transient thermography technique with a heating or excitation duration in the specified range, instead of flash thermography, to radiatively heat the composite structure 12 allows sufficient thermal energy to penetrate into the composite structure 12 despite the layer of paint 16 and even presence of the polymer film 18. Accordingly, the composite structure 12 may be radiatively heated to a depth of between about 550 microns (μm) and about 950 μm. The composite structure 12 may thus be sufficiently heated to reveal thermal anomalies in the composite structure 12 regardless of paint thickness and presence of the polymer film 18.

The composite structure 12 may be radiatively heated with a radiative source 20 having a radiative power of between about 100 watts (N) and 500 W. The radiative source or radiative heating source 20 may comprise one or more low power radiative heating lamps. Examples of low power radiative heating lamps include, but are not limited to, an infrared lamp, a halogen lamp and a light emitting diode (LED). Advantageously, the use of the low power radiative source, which may be powered by a portable battery source, increases the convenience and maneuverability of the method 10 as it allows for the method 10 to be used with a portable active infrared thermography (IRT) system that does not require connection to a wall socket via a power cable.

Unlike convective heat transfer, radiative heat transfer allows for the radiative source 20 to be placed at a further distance from the composite structure 12. Accordingly, the radiative source 20 may be positioned at a distance D of between about 0.15 metres (m) and about 0.80 m from a surface 22 of the composite structure 12.

At step 24, the heated composite structure 12 is cooled.

Temperature changes of the composite structure 12 are monitored at step 26 as the composite structure 12 cools. The temperature changes of the composite structure 12 may be monitored with a thermal camera 28. An infrared spectrum may be divided into three bands that correspond to three atmospheric transmission windows: short-wave infrared (SWIR) spectral band of approximately 1 μm to 2.5 μm, mid-wave infrared (MWIR) spectral band of approximately 3 μm to 5 μm and long-wave infrared (LWIR) spectral band of approximately 7 μm to 14 μm. In embodiments of the present invention, the thermal camera 28 may be a cooled mid-wave (3 μm to 5 μm wavelength range) thermal camera or an uncooled long-wave (8 μm to 15 μm wavelength range) thermal camera. In preferred embodiments, the thermal camera 28 may include an uncooled detector and may be operable in a long-wave infrared (LWIR) spectral band of between about 8 μm and about 15 μm as an uncooled long-wave thermal camera is less costly. The thermal camera 28 may be provided with a visible camera or may be without.

By using radiative heating instead of conventional heating, this allows for heating of the composite structure from a further distance and this makes it possible to have the radiative source 20 and the thermal camera 28 at the same distance. Accordingly, in one or more embodiments, the radiative source 20 and the thermal camera 28 may both be positioned substantially equidistant from the surface 22 of the composite structure 12. Advantageously, this allows integration of the radiative source 20 and the thermal camera 28 into a portable active infrared thermography (IRT) system, making it possible for inspection to be conducted by a single inspector, instead of the current practice of having two inspectors.

At step 30, a thermal image of the composite structure is generated based on the temperature changes. The thermal image corresponds to thermal gradients due to temperature differences between defective and non-defective areas of the composite structure 12. The location of defects is detected by the thermal camera 28 through a process of mapping temperature distribution on the surface 22 of the composite structure 12.

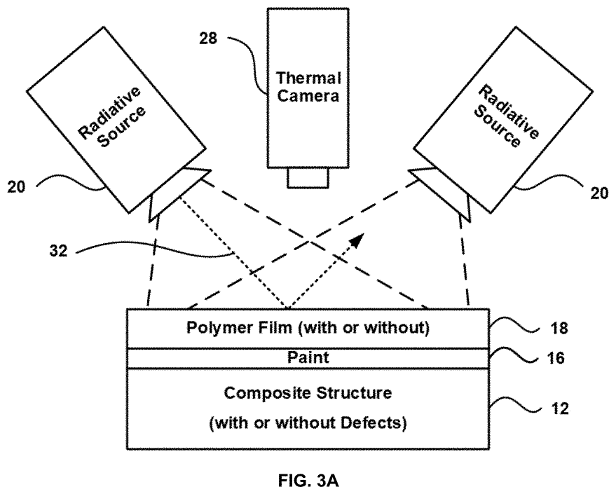

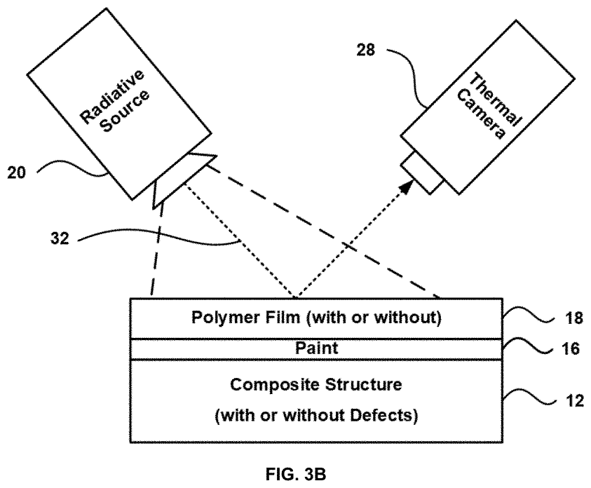

Referring now to FIGS. 2, 3A and 3B, to minimise high thermal noise or the appearance of artefacts on the thermal image due to stray heat from the low power radiative source 20 interfering with one or more signals captured by the thermal camera 28, the thermal camera 28 may be positioned outside a path of reflected heat 32 from the radiative source 20 as shown in FIGS. 2 and 3A, in contrast to FIG. 3B showing the thermal camera 28 in the path of reflected heat 32 from the radiative source 20. This allows integration of the radiative source 20 and the thermal camera 28 in spite of the different form factors (for example, lamp diameter) of the low power radiative source 20 (for example, one or more infrared lamps, one or more halogen lamps and/or one or more LEDs).

### EXAMPLES

### Example 1

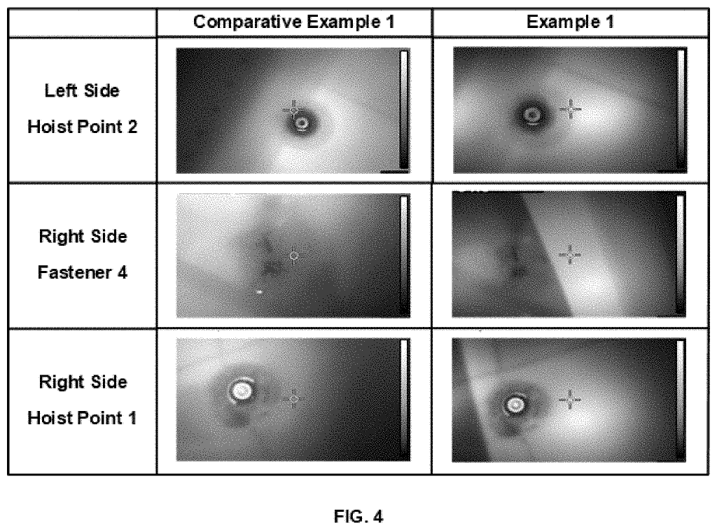

An actual on-site inspection was conducted on 14 Oct. 2019 using (1) a comparative method employing an air heater to heat up a composite surface and a thermal camera to monitor changes of temperature during cooling (“Comparative Example 1”), and (2) the method 10 for detecting a thermal anomaly in a composite structure described above (“Example 1”). A comparison of thermal images obtained from both methods is shown in FIG. 4. One advantage of the method 10 described above over the comparative method is that the latter requires two inspectors, whereas the former may be performed with only a single inspector.

### Example 2

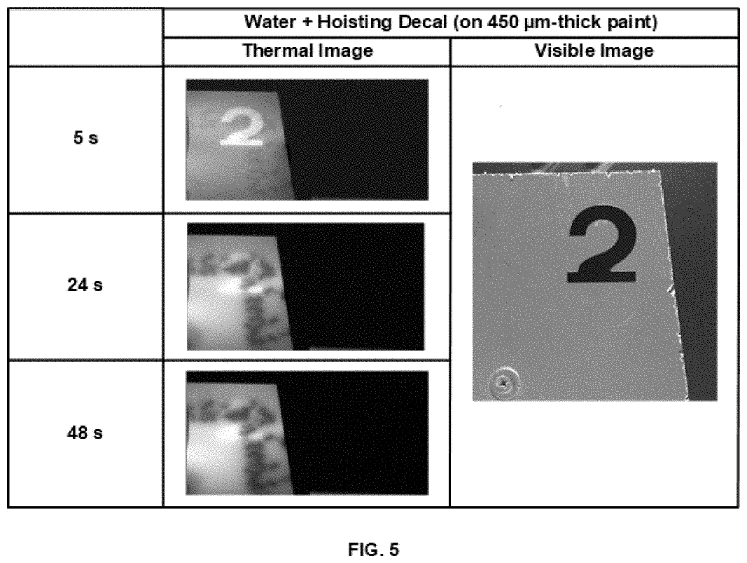

Referring now to FIG. 5, the effect of different heating durations (5 s, 24 s and 48 s) on the thermal signature of an aircraft honeycomb composite surface with a hoisting decal on top is shown. An approved active IRT setup was used to study the effect of heating duration on thermal signature. The distance of the radiative source to the sample surface was kept at 15 centimetres (cm) and the distance of the thermal camera to the sample surface was kept at 80 cm. The thermal signature is due to artificially created water ingress underneath the sample surface. The thermal anomalies (dark in colour) are due to water ingress underneath the sample surface.

The study demonstrated that if the heating duration is too short (i.e. 5 s), the thermal wave will not be able to penetrate the hoisting decal and thus thermal anomalies under the hoisting decal will not be detected.

Conversely, if the heating duration is too long (i.e. 48 s), the thermal signature will be “blurred out” and this might lead to an over-estimate of the extent of water ingress underneath the sample surface.

It was therefore concluded based on the experiment that a heating duration of approximately 24 s will give the best results.

### Example 3

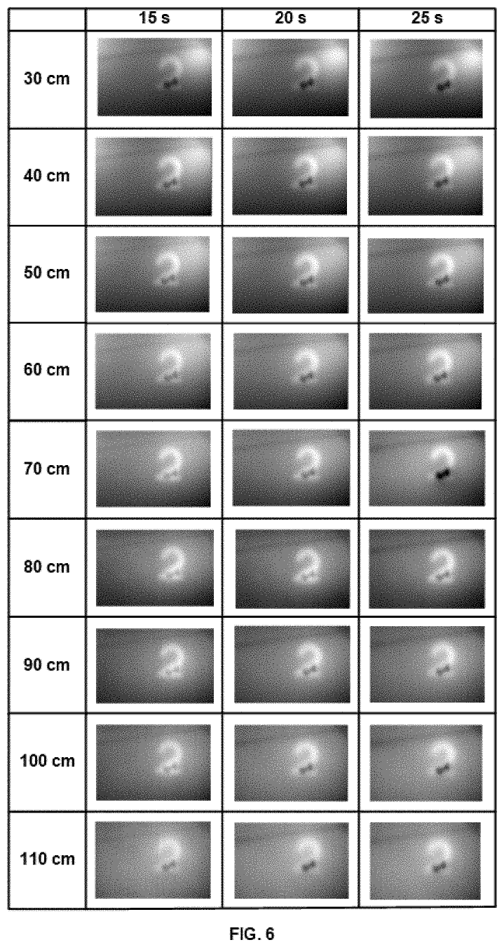

Referring now to FIG. 6, the effect of heating duration (15 s, 20 s and 25 s) and distance (30 cm to 110 cm, in increments of 10 cm) on the thermal signature of an aircraft honeycomb composite surface with a hoisting decal on top is shown. The aircraft honeycomb composite includes a paint thickness of 750 μm with a hoisting decal on top. The thermal signature is due to artificially created water ingress underneath the sample surface. The thermal anomalies (dark in colour) are due to water ingress underneath the sample surface. The aim of this experiment is to evaluate the active IRT configurations under a worst scenario of water ingress under thick (750 μm) paint and hoisting decal. Defect detection in such a scenario would not have been possible using a high-power flash lap to heat up the composite surface.

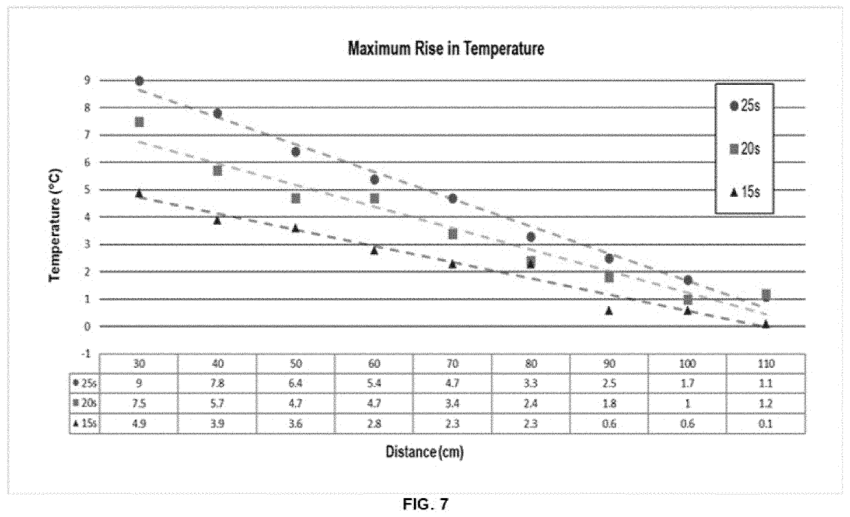

Referring now to FIG. 7, a plot of maximum rise in temperature as a function of heating duration and distance is shown. Data were obtained from the radiometric images of FIG. 6 and the maximum rise in temperature was calculated from the difference between the maximum temperature and the ambient temperature.

As can be seen from FIG. 7, the maximum rise in temperature decreases with decreasing heating duration and increasing distance from the sample surface. As the rise in temperature is significant (i.e. 2° C. in this case) for distances not exceeding 80 cm from the surface, this warrants a good thermal contrast between the defects and the surrounding matrix for all 3 sets of heating duration (15 s, 20 s and 25 s).

As is evident from the foregoing discussion, the present invention provides an active infrared thermography method for detecting thermal anomalies in composite structures that allows greater flexibility in positioning of components and that can also overcome composite surface restrictions. Advantageously, the use of a low-power (less than 500 W) transient-thermography technique instead of high-power flash-thermography technique allows heat to penetrate thick paint (750 μm) over which a polymer film in the form of a hoisting decal may be applied.

While preferred embodiments of the invention have been described, it will be clear that the invention is not limited to the described embodiments only. Numerous modifications, changes, variations, substitutions and equivalents will be apparent to those skilled in the art without departing from the scope of the invention as described in the claims. The method of the present invention may be used in aircraft maintenance applications such as, for example, rudder inspection.

Further, unless the context clearly requires otherwise, throughout the description and the claims, the words “comprise”, “comprising” and the like are to be construed in an inclusive as opposed to an exclusive or exhaustive sense; that is to say, in the sense of “including, but not limited to”.

1. A method for detecting a thermal anomaly in a composite structure, comprising:
radiatively heating the composite structure for a period of between about 15 seconds (s) and about 25 s;
cooling the heated composite structure;
monitoring temperature changes of the composite structure as the composite structure cools; and
generating a thermal image of the composite structure based on the temperature changes.

2. The method of claim 1, wherein the composite structure is radiatively heated to a depth of between about 550 microns (μm) and about 950 μm.

3. The method of claim 1, wherein the composite structure is radiatively heated with a radiative source having a radiative power of between about 100 watts (W) and 500 W.

4. The method of claim 3, wherein the radiative source is positioned at a distance of between about 0.15 metres (m) and about 0.80 m from a surface of the composite structure.

5. The method of claim 3, wherein the temperature changes of the composite structure are monitored with a thermal camera and wherein the thermal camera is positioned outside a path of reflected heat from the radiative source.

6. The method of claim 5, wherein the radiative source and the thermal camera are both positioned substantially equidistant from the surface of composite structure.

7. The method of claim 5, wherein the thermal camera comprises an uncooled detector and wherein the thermal camera is operable in a long-wave infrared (LWIR) spectral band of between about 8 μm and about 15 μm.
